(19) United States
(12) Patent Application Publication
(10) Pub. No.: US 2014/0073612 A1
(43) Pub. Date: Mar. 13, 2014

(54) TRANSDERMAL DELIVERY SYSTEM KIT

(71) Applicants: Carlos A. Alfaras, Miami, FL (US); Paul M. Zimmerman, Miami Beach, FL (US); Urbano Zamora, Plantation, FL (US)

(72) Inventors: Carlos A. Alfaras, Miami, FL (US); Paul M. Zimmerman, Miami Beach, FL (US); Urbano Zamora, Plantation, FL (US)

(21) Appl. No.: 13/609,810

(22) Filed: Sep. 11, 2012

(51) Int. Cl. A61M 35/00; A61P 29/00; A61K 31/165; A61P 35/00; A61K 31/192; A61K 31/522; A61K 31/4418; A61P 21/02

(52) U.S. Cl. 514/162; 514/356; 514/570; 514/263.34; 514/627; 604/304; 604/307

(57) ABSTRACT

A transdermal delivery kit that enhances the efficacy of a topical drug includes at least one drug dispensing device, a lipophilic base, a hydrophilic base, a lipid-soluble active ingredient, a water-soluble active ingredient, a first container for mixing the lipophilic base with the lipid-soluble active ingredient to form a first compound, a second container for mixing the hydrophilic base with the water-soluble active ingredient to form a second compound, a mixing member, and instructions for compounding the lipophilic base with the lipid-soluble active ingredient to form the first compound, compounding the hydrophilic base with the water-soluble active ingredient to form the second compound, and mixing the first compound and the second compound to form a transdermal pharmaceutical delivery system capable of delivering a drug to the dermal layer of the skin.

### FIELD OF THE INVENTION

The present invention relates generally to a transdermal delivery system kit. More so, the present invention provides a kit for enhancing the efficacy of a transdermal drug by combining accurate and convenient compounding of the transdermal drug with efficient delivery through a transdermal delivery carrier.

### BACKGROUND OF THE INVENTION

The following background information may present examples of specific aspects of the prior art that, while expected to be helpful to further educate the reader as to additional aspects of the prior art, is not to be construed as limiting the present invention, or any embodiments thereof, to anything stated or implied therein or inferred thereupon.

Typically, drugs are compounded by a pharmacist to fit the unique needs of a patient. To compound the drug, pharmacists combine and/or process appropriate ingredients utilizing various tools. This may be done for medically necessary reasons, such as to change the form of the medication from a solid pill to a liquid, to avoid a non-essential ingredient that the patient is allergic to, or to obtain the exact dose needed. Another reason a pharmacist may need to compound a drug is to avoid stability issues of the final compounded drug product. Some compounded medications have a very short shelf life and, therefore, have many stability issues making the final compounded product not cost effective to manufacture well in advance. Compounding may also be done for voluntary reasons, such as adding favorite flavors to a medication.

In many instances, a drug's efficacy may be affected by the manner in which it is compounded and then administered into the body. Drugs may be prepared, mixed, assembled, and packaged exactly as a prescription demands, yet the patient may not have the capacity to properly administer the drug. Many conditions may require sustained or repeated delivery of drugs to a specific area of the body. However, systemic delivery may be limited by transfer through the membrane, thus requiring very high systemic doses that may lead to toxicity.

Typically, drug delivery is the method or process of administering a pharmaceutical compound to achieve a therapeutic effect in humans or animals. Drug delivery technologies modify drug release profile, absorption, distribution and elimination for the benefit of improving product efficacy and safety, as well as patient convenience and compliance. Drug release is from: diffusion, degradation, swelling, and affinity-based mechanisms. Most common routes of administration include the preferred non-invasive peroral (through the mouth), topical (skin), transmucosal (nasal, buccal/sublingual, vaginal, ocular and rectal) and inhalation routes.

Typically, transdermal delivery is a route of administration wherein active ingredients are delivered across the skin through the epidermis and into the dermal layer for systemic distribution into the bloodstream and internal organs. More specifically, transdermal delivery refers to the route of administration wherein active ingredients are delivered across the skin, and then pass through two sublayers of epidermis to reach the microcirculation of the dermis for systemic distribution. It is significant to note that simply applying a medication topically does not mean the medication will reach the microcirculation of the dermis and have a systemic effect. Examples of transdermal delivery methods include transdermal patches used for medicine delivery, and transdermal implants used for medical or aesthetic purposes. Transdermal drug delivery typically involves a carrier, such as a liquid, gel, solid matrix, or pressure sensitive adhesive into which the drug to be delivered is incorporated. The drug containing carrier is then placed on the skin and the drug, along with any adjuvants and excipients is delivered to the skin.

A transdermal patch is a medicated adhesive patch that is placed on the skin to deliver a specific dose of medication through the skin and into the bloodstream. Often, this promotes healing to an injured area of the body. An advantage of a transdermal drug delivery route over other types of medication delivery such as oral, topical, intravenous, intramuscular, etc. is that the transdermal patch provides a controlled release of the medication into the patient, usually through either a porous membrane covering a reservoir of medication or through body heat melting thin layers of medication embedded in the adhesive. Another advantage of transdermal delivery systems is the bypass of absorption through the stomach. This bypass may decrease possible esophageal and gastrointestinal lining deterioration issues and possible ulcerations which are well-known complications of long term oral non-steroidal anti-inflammatory drug use. Another main advantage of transdermal delivery systems is any active drug that has a low oral bio-availability will pass the “first pass metabolism” through the liver, thus greatly increasing the drugs bio-availability to the patient. The main disadvantage to transdermal delivery systems stems from the fact that the skin is a very effective barrier; as a result, only medications whose molecules are small enough to penetrate the skin can be delivered by this method.

Even though the above cited transdermal delivery systems and methods address some of the needs of the market, a kit for enhancing the efficacy of a transdermal drug by combining accurate compounding of the transdermal drug, and efficient delivery through a transdermal delivery carrier is still desired.

### SUMMARY OF THE INVENTION

The invention provides a transdermal delivery system kit that overcomes the hereinafore-mentioned disadvantages of the heretofore-known devices and methods of this general type and that enhances the efficacy of a topical drug by facilitating the accurate compounding of the drug, and then providing a transdermal delivery carrier for efficient administration of the drug. In some embodiments, the transdermal delivery system kit provides a prepackaged environment that is efficacious for the accurate compounding of at least one active agent and at least one inactive agent. The resultant drug may then be stored into at least one drug dispensing device for future dispensing, for example, onto at least one occlusive member for systemic transdermal administration. In some embodiments, the initial drug compounding may be performed by a pharmacist who utilizes the transdermal delivery system kit to compound the drug from each active and each inactive ingredient in the transdermal delivery system kit. A variety of tools may be utilized to facilitate the compounding process, including, without limitation, a mixing member, instructions for compounding, inactive agents, bases, active agents, pH actuators, and solvents. However, those skilled in the art will recognize that additional agents and tools may be included in the transdermal delivery system kit to compound myriad of other drugs. In some embodiments, at least one inactive agent is prepackaged and premeasured into at least one drug dispensing device. Those skilled in the art, in light of the present teachings will recognize that the inactive agent may include a permeation enhancer for enhancing absorption of the drug through the top layer of the skin, which is the stratum corneum, then through the epidermis, and finally to the dermal layer which lies below the epidermis before delivering a specific dose of the drug into the bloodstream, joint, or affected area. The inactive agent may include, without limitation, solvents, anionic surfactants, nonionic surfactants, and essential oils. Other inactive agents commonly utilized for compounding drugs may include, without limitation, pH actuators, speed gels, and the like. The inactive agent may also include a permeation enhancer for enhancing absorption of the drug locally, i.e., not systemically absorbed.

At least one active agent may also be prepackaged and premeasured in at least one container, such as a premeasured glass vial. The active agents may then be compounded with the inactive agents to form the desired drug per instructions included in the transdermal delivery system kit. A support member may be utilized to support and organize individual foil packets of the various agents, pH actuators, and solvents. In some embodiments, the drug may include a topical ointment, including, without limitation, a gel, a cream, oil, white petroleum, and the like. At this point, additional components of the transdermal delivery system kit may be utilized to administer the drug.

In some embodiments, a patient may dispense the drug from the at least one drug dispensing device. The at least one drug dispensing device may include, without limitation, a pump dispenser, a dropper, or a medicine bottle. The drug may be dispensed onto at least one occlusive member from the transdermal delivery system kit. In another embodiment, the drug may be applied directly to the skin, and then optionally covered with an occlusive patch. Each occlusive member may be efficacious for actuating absorption of the drug through the topical contact surface area. The absorption initiates through the stratum corneum, then through the epidermis, and finally to the dermal layer before delivering a specific dose of the drug into the bloodstream, joint, or affected area. The occlusive member may actuate absorption in a variety of ways. First, the occlusive member may act as a reservoir for the drug, releasing the drug in a staged, time-release manner. In another system, the occlusive member acts as a cover over the skin. The covering effect may help to drive medication into the skin with the help of body heat. It is well known in the art that retaining moisture from the top layer of the skin enhances absorption. In yet another system the occlusive member simply acts as a cover to prevent the drug from rubbing off. Each occlusive member may include, without limitation, a transdermal delivery patch and a bandage. Each occlusive member may include a contact surface and an opposite external surface. The drug may be dispensed onto the contact surface, and the contact surface may be positioned over the desired topical contact surface area. The contact surface may then be maintained over the topical contact surface area for a predetermined quantity of time, or until absorption of the drug through the topical contact surface area is complete. Additional embodiments of the occlusive member may include two separate components: 1) a drug-saturated member for absorbing the drug prior to use and 2) an adhesive member for adhering the drug saturated member onto the topical contact surface area when needed.

With the foregoing and other objects in view, there is provided, in accordance with the invention, a transdermal delivery system kit for compounding a drug and administering the drug through transdermal delivery, where the kit includes at least one drug dispensing device, a lipophilic base, a hydrophilic base, a lipid-soluble active ingredient, a water-soluble active ingredient, a first container for mixing the lipophilic base with the lipid-soluble active ingredient to form a first compound, a second container for mixing the hydrophilic base with the water-soluble active ingredient to form a second compound, a mixing member, and instructions for compounding the lipophilic base with the lipid-soluble active ingredient to form the first compound, compounding the hydrophilic base with the water-soluble active ingredient to form the second compound, and mixing the first compound and the second compound to form a transdermal pharmaceutical delivery system capable of delivering a drug to the dermal layer of the skin.

In accordance with another feature, an embodiment of the present invention includes at least one occlusive member for actuating absorption of the drug through a topical contact surface area of the recipient of the drug.

In accordance with a further feature of the present invention, the at least one occlusive member comprises a transdermal delivery-patch.

In accordance with yet another feature of the present invention, the at least one occlusive member includes a topical contact surface, the topical contact surface of the occlusive member being configured to maintain the drug in contact with the topical contact surface area.

In accordance with one more feature of the present invention, the at least one drug dispensing device comprises a pump dispenser.

In accordance with a further feature of the present invention, the at least one drug dispensing device is calibrated to dispense a predetermined quantity of the drug.

In accordance with an additional feature of the present invention, the at least one of the lipid-soluble active ingredient, the water-soluble active ingredient, the lipophilic base, and the hydrophilic base are provided in a quantity that can be used to form the drug without the need for measurement by a compounding user.

In accordance with another feature of the present invention, the at least one of the lipid-soluble active ingredient and the water-soluble active ingredient comprise an analgesic, an anti-inflammatory, a muscle relaxer, a biologically active protein, a cellulite reducer, a substance P antagonist, or an antineoplastic compound.

In accordance with a one more feature of the present invention, the at least one of the lipophilic base and the hydrophilic base comprise a biocompatible organic solvent, a polar lipid, a surfactant, water, urea, a suspending agent, or an anti-foaming agent.

In accordance with a further feature of the present invention, the at least one of the lipophilic base and the hydrophilic base comprise a permeation or penetration enhancer for transdermal drug delivery.

In accordance with the present invention, a method for compounding a transdermal delivery system with a transdermal delivery system kit includes the step of providing at least one drug dispensing device, a lipophilic base, a hydrophilic base, a lipid-soluble active ingredient, a water-soluble active ingredient, a first container for mixing the lipophilic base with the lipid-soluble active ingredient to form a first compound, and a second container for mixing the hydrophilic base with the water-soluble active ingredient to form a second compound. The method further includes the steps of compounding the lipophilic base with the lipid-soluble active ingredient to form the first compound, compounding the hydrophilic base with the water-soluble active ingredient to form the second compound, and mixing the first compound and the second compound to form a transdermal pharmaceutical delivery system capable of delivering a drug to the dermal layer of the skin.

These and other advantages of the invention will be further understood and appreciated by those skilled in the art by reference to the following written specification, claims, and appended drawings.

Although the invention is illustrated and described herein as embodied in a transdermal delivery system kit, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

### BRIEF DESCRIPTION OF THE DRAWINGS

The invention will now be described, by way of example, with reference to the accompanying drawings, in which:

FIG. 1 presents a detailed perspective view of an exemplary transdermal delivery system kit, in accordance with an embodiment of the present invention;

FIG. 2 presents an elevated side view of an exemplary container dispersing at least one active agent into an exemplary drug dispensing device, in accordance with an embodiment of the present invention;

FIG. 3 presents a perspective view of an exemplary occlusive member folded to expose its application surface and receiving an exemplary drug from the drug dispensing device, in accordance with an embodiment of the present invention;

FIG. 4 presents a detailed perspective view of the occlusive member covering an exemplary topical contact surface area, in accordance with an embodiment of the present invention;

FIGS. 5A & 5B are an elevational cross-sectional views of an occlusive member, e.g., a transdermal delivery patch and a bandage, in accordance with an embodiment of the present invention;

FIG. 6 is a perspective view of a packaged transdermal delivery system kit, in accordance with embodiment of present invention;

FIG. 7 is a perspective partially hidden view of the transdermal delivery system kit of FIG. 6;

FIG. 8A is an exploded perspective view of the transdermal delivery system kit of FIG. 6 showing individually-packaged premeasured active drugs in an expanded view;

FIG. 8B is an exploded perspective view of the transdermal delivery system kit of FIG. 8A showing the individually-packaged premeasured active drugs in a columnar arrangement for insertion within the kit housing in accordance with an embodiment of the present invention;

FIG. 9 is a close-up perspective view of a stacked column with sections of packages containing premeasured quantities of active ingredients, in accordance with an embodiment of the present invention; and

FIG. 10 is a process flow diagram illustrating a method for creating a transdermal delivery system in accordance with an embodiment of the present invention.

### DETAILED DESCRIPTION OF THE INVENTION

The following detailed description is merely exemplary in nature and is not intended to limit the described embodiments or the application and uses of the described embodiments. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

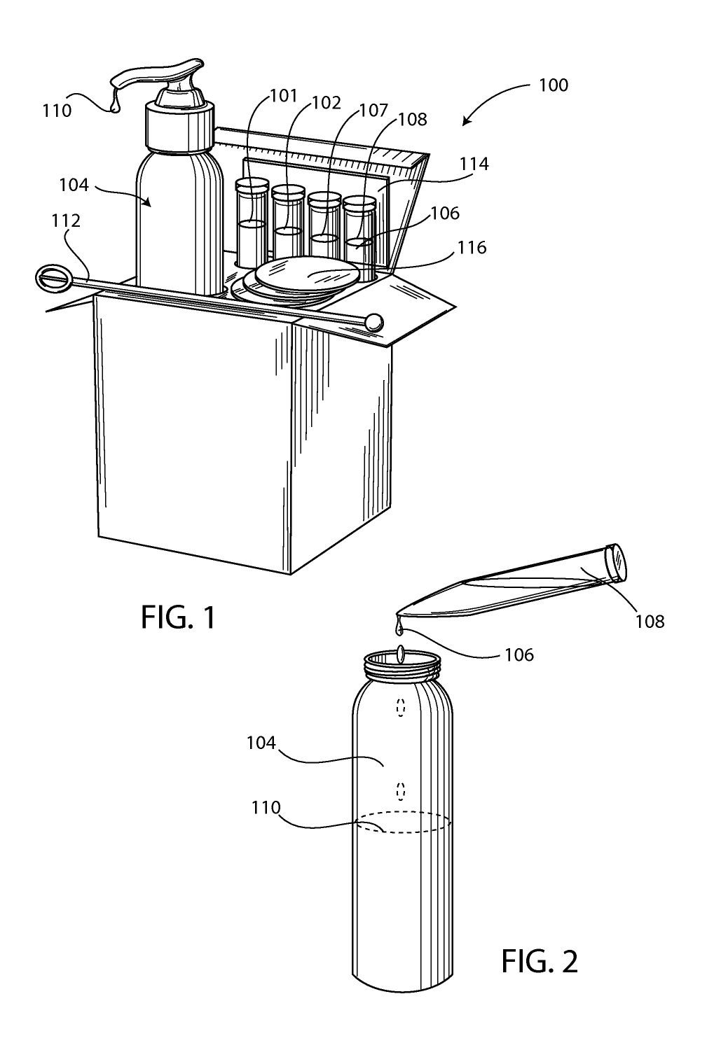

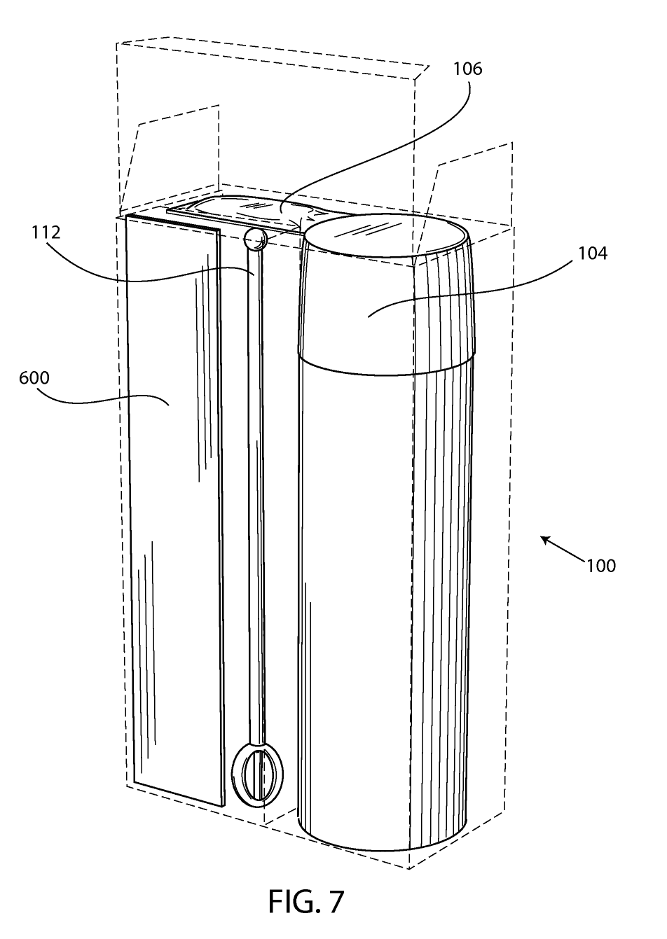

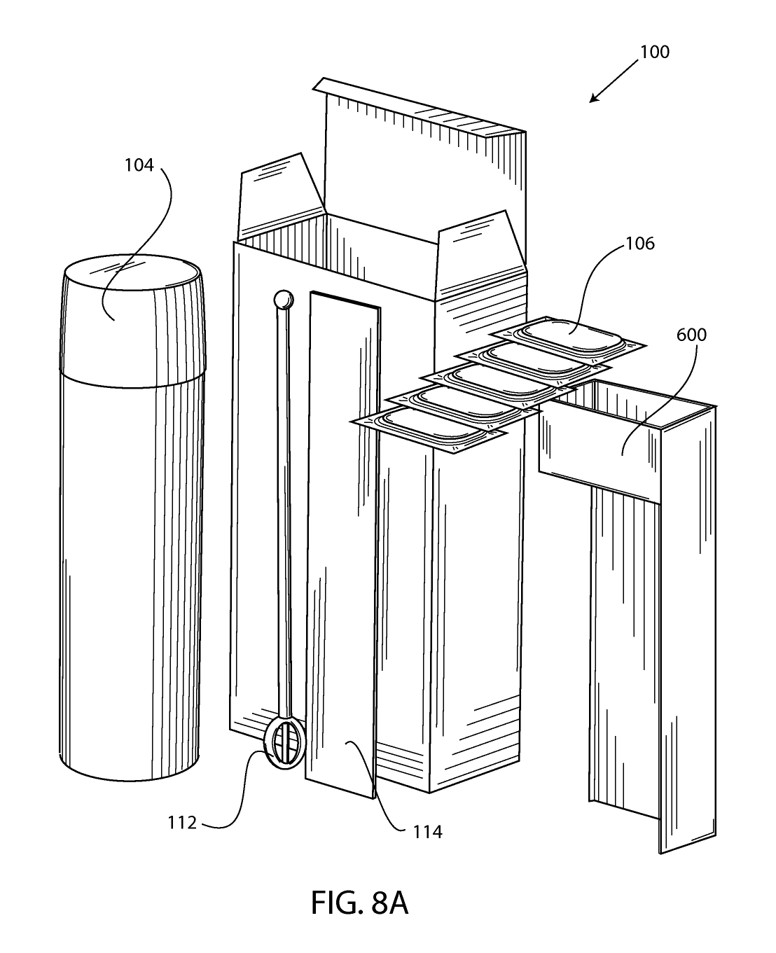

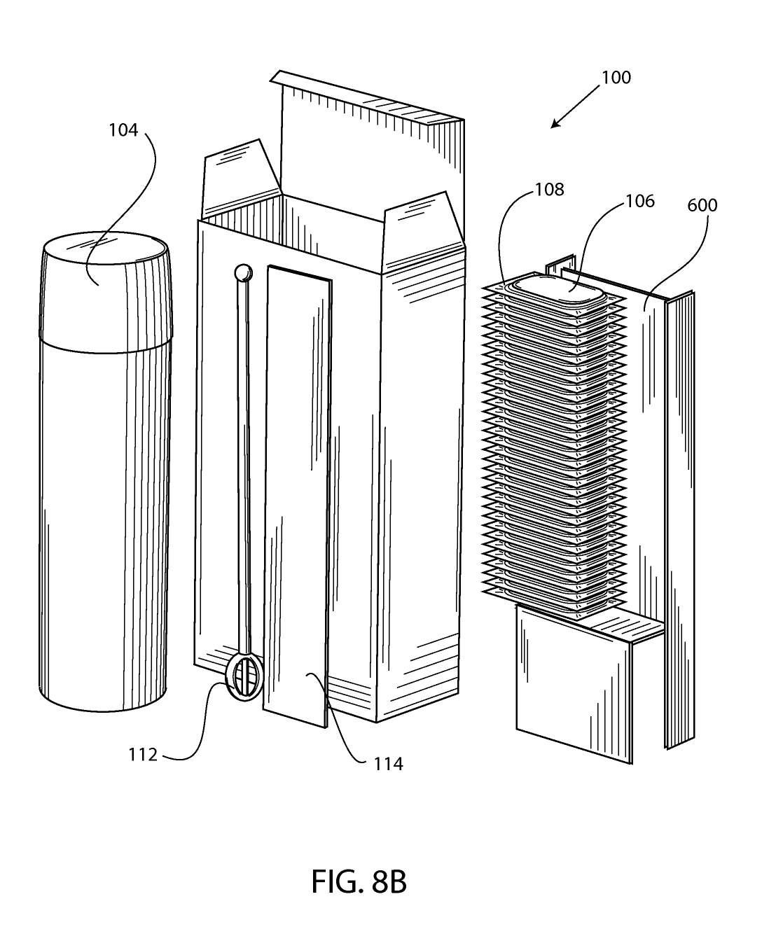

A transdermal delivery system kit 100 is described in FIGS. 1 through 9. The transdermal delivery system kit 100 is a system that may include: At least one drug dispensing device 104 for dispensing a compounded drug 110. In some embodiments, the compounded drug 110 may include a topical ointment that is conducive to transdermal delivery, including, without limitation, a gel, a cream, oil, and the like. Specific embodiments of the drug 110 may include, without limitation, Fentanyl, Nitroglycerine, Nicotine, Testosterone, Clonidine, Lidocaine, Scopolamine, Cyclobenzaprine, and Ethinyl Estradiol. The drug 110 may be efficacious in treating various maladies including, without limitation, arthritis, hemorrhoids, hormone deficiency, nicotine replacement, contraceptive inducement, muscle spasms, pain relief, inflammation and the like. Those skilled in the art, in light of the present teachings, will recognize that the efficacy of the drug 110 may be affected by the manner in which the drug 110 is compounded and then administered, as well as the properties of the inactive ingredients used. The drug 110 may be prepared, mixed, assembled, and packaged exactly as a prescription demands. Further, the drug 110 in the present invention can be effective for both treating the malady and transdermal delivery.

In some embodiments, at least one drug dispensing device 104 may be utilized to dispense the final compounded drug 110. An exemplary drug dispensing device 104 is presented in FIGS. 1, 2, 3, 6, 7, 8A and 8B. The at least one dispensing device 104 may include, without limitation, a pump dispenser, a dropper, or a medicine bottle. In one embodiment, the at least one drug dispensing device 104 may include a calibrated pump that dispenses a predetermined quantity of the drug 110. Each drug dispensing device 104 may include at least one inactive agent 110, and usually two, in a predetermined quantity. Each inactive agent may include, without limitation, a biocompatible organic solvent, a polar lipid, a surfactant, a suspending agent, an anti-foaming agent, water, and urea. Those skilled in the art, in light of the present teachings, will recognize that transdermal delivery of the drugs 110 often requires use of a permeation enhancer to assist in the delivery of the drug 110. One such permeation enhancer can be certain solvents; whereby the solvent may include a liquid, solid, or gas that dissolves another solid, liquid, or gaseous solute, resulting in a solution that is soluble in a certain volume of solvent at a specified temperature. Solvents that are well known in the art for enhancing permeation include, without limitation, methanol, ethanol, dimethyl sulfoxide, propylene glycol, 2-pyrrolidone, isopropyl myristate, and laurocapram (Azone). An anionic surfactant such as sodium lauryl sulfate may also be utilized in the compounding process to act as a permeation enhancer. Yet another permeation enhancer may include nonionic surfactants, such as, without limitation, sorbitan, monolaurate, and pluronic. In some embodiments, oil may be easily incorporated in topical preparations as an inactive agent since oils are stable in water emulsions, thereby enhancing permeation of drugs through the skin. Some of these oils may include, without limitation, cardamom oil, caraway oil, lemon oil, menthol, d-limonene, and linoleic acid. Those skilled in the art will recognize that the solvents, surfactants, and oils may be used in combination, or separately to compound the drug 110.

The transdermal delivery system kit 100 may include at least one compounded drug dispensing device 104 and a first inactive agent or base 101, which is a lipid base. The transdermal delivery system kit 100 may also include at least one second inactive agent or base 102, which is a water/urea base. Further, the transdermal delivery system kit 100 can include a first active ingredient 106 that is water soluble and a second active ingredient 107 that is lipid soluble, although both are not required.

Each container 108 may be a premeasured medicine vial that stores a premeasured quantity of the active agent(s) 106, 107 or inactive agent(s) 101, 102. In some embodiments, the active agents 106, 107 may include, without limitation, an analgesic, an anti-inflammatory, a biologically active protein, a cellulite reducer, a substance P antagonist, or an antineoplastic compound.

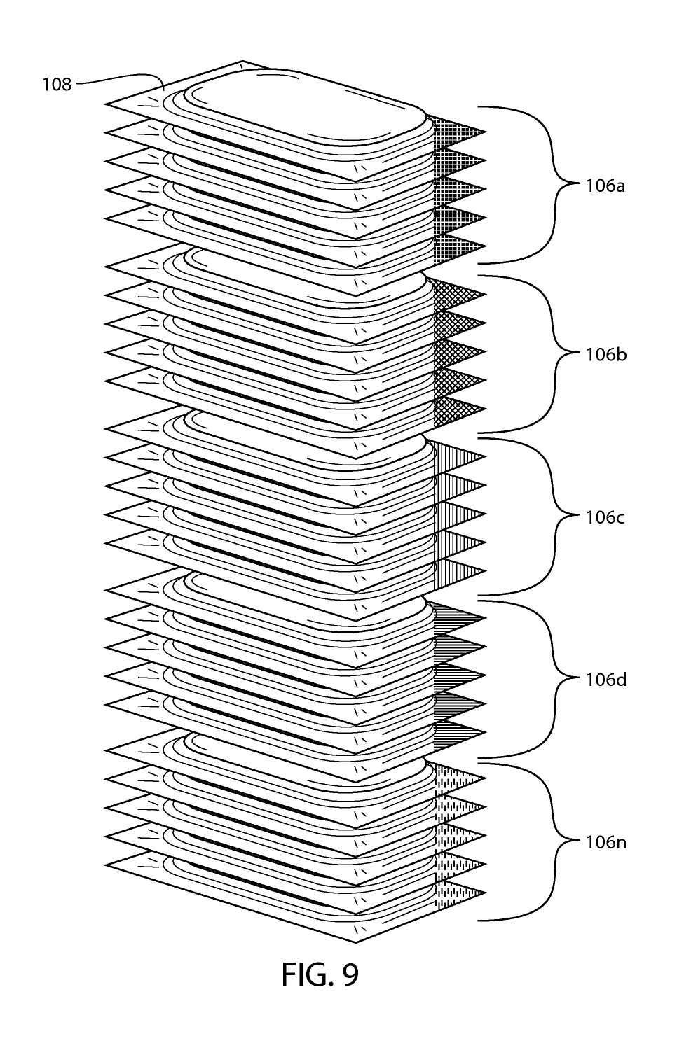

FIG. 2 presents an elevated side view of an exemplary container dispersing at least one active agent 106 into an exemplary drug dispensing device 104. Those skilled in the art, in light of the present teachings will recognize that compounding the drug 110 may include instructions 114 (shown in FIG. 1) for directing the compounding process, including, without limitation, directions for mixing the active agents 106, 107 and bases 101, 102 and adding them to the drug dispensing device 104, directions for adjusting the pH, directions for utilizing thermal energy during mixing, directions for adding solvents, suspending agents, surfactants, and the like. A mixing member 112 may then be utilized to further enhance the drug compounding process. The mixing member 112 may include a spatula, a stirring stick, a source of thermal energy. In some embodiments, the resultant drug 110 may include a topical ointment, including, without limitation, a gel, a cream, oil, white petroleum, and the like. In some embodiments, the active agent(s) 106, 107 may be contained in individual packets. FIG. 9 shows multiple exemplary packets of possible active agents. The active agent(s) 106, 107 may be in the form of, without limitation, powder form, crystal form, or liquid form.

In one embodiment, the transdermal delivery system kit 100 includes five inactive ingredients, with three of the five being lipid-based inactive ingredients and two are water-based inactive ingredients. The kit 100 also includes two active ingredients, with the first being water/urea soluble and the second being lipid soluble. Examples of these ingredients are recited in commonly-owned U.S. Pat. No. 5,654,337. In a first step, the three lipid based ingredients are mixed together to create a compound “TD1.” Next, the two water-based ingredients are mixed together to create a compound “TD2.” The lipid-soluble active ingredient 106 is added to TD1 and the water/urea-soluble active ingredient is added to TD2. Finally, all the ingredients are added to container 104 and mixed together thoroughly. In one embodiment, the bases/inactive agents 101, 102 may include, without limitations a speed gel, a gel, an ointment, a surfactant, and more.

The bases/inactive agents 101, 102 and active ingredients 106, 107 are premeasured into the containers 108 which can be, for example, glass vials. In other embodiments, as shown in FIG. 6, the active ingredients 106, 107 are provided within packet-type containers that can be opened and added to mixing containers, such as the tubes 108. Those skilled in the art will recognize that the transdermal delivery system kit 100 may include a support member 600 (shown in FIG. 6) to organize and support individual packets of the active agents 106, 107. The support member may include a rigid channel sized and dimensioned to hold the active agents 106, 107. In some embodiments, the active agent/inactive agent compound may be compounded inside the at least one dispensing device 104 with the base to form the drug.

Those skilled in the art, in light of the present teachings will recognize that a myriad of drugs 110 may be compounded with the transdermal delivery system kit 100. In some embodiments, each active agent 106, 107 and each inactive agent 101, 102 may be compounded to form a topical cream, gel, liquid, or powder efficacious for transdermal delivery through a topical contact surface area 400. For example, without limitation:

### Example 1

### Preparation of Speed Gel

| | 360 gm | |
|---|---|---|
| *LID Oil | 36 | gm |
| **Lecithin organogel (L.O.) | 72 | gm |
| Docusate sodium powder | 36 | gm |
| Urea | 36 | gm |
| Distilled water | 180 | ml |

*LID oil is a 1:1 mixture of lecithin, isopropyl, and docusate.
**L.O. is a mixture of lecithin and isopropyl myristate.

1. The LID was added to L.O. and heated.
2. Docusate sodium powder was added, and the mixture was stirred until smooth.
3. Urea was added to water, heated, and added to step 2 with stirring.
4. pH was adjusted to between 6.5 to 6.9.

Speed-gel may just as easily be prepared as follows:

| | 100 gm |
|---|---|
| L.O. | 25 gm |
| Docusate sodium benzoate powder | 15 gm |
| Urea | 10 gm |
| Distilled water | 50 gm |

The L.O. was heated and the docusate sodium benzoate powder was stirred into the heated L.O. until a smooth solution is prepared. The water was heated and the urea was dissolved into the water, and the urea solution was then thoroughly mixed with the docusate sodium containing solution of L.O. The result was a consistent, transparent, amber colored gel with a pH of about 6.0.

Yet another way of making speed-gel is as follows:

| | 100 gm |
|---|---|
| L.O. | 10 gm |
| L.I.D. | 30 gm |
| Urea | 10 gm |
| Distilled water | 50 gm |

The L.O. and L.I.D. were mixed well, and a heated solution of water and the urea was prepared and added to the L.I.D.-L.O. solution. The result was a consistent, transparent, amber colored gel with a pH of about 6.0.

### Example 2

### Preparation of Nifedipine Composition

| | 360 gm | |
|---|---|---|
| Nifedipine | 0.3 | gm |
| Dimethyl sulfoxide (DMSO) | 43 | drops |
| Polysorbate 80 | 2 | ml |
| Speed-gel | 30 | gm |

1. Nifedipine was dissolved in the DMSO with trituration 60 in mortar.
2. 2 ml polysorbate 80 was added with trituration to thicken.
3. 30 gm with new speed gel.
The composition was dispensed into a 1 oz. cc syringe with Luer tip cap and stored in a light-resistant bag.

### Example 3

### Preparation of 5% Ketoprofen Composition

| | 30 gm | |
|---|---|---|
| Ketoprofen | 1.5 | gm |
| Lecithin organogel | 5 | gm |
| Speed-gel | 30 | gm |

The L.O. was heated and ketoprofen added and stirred until a consistent, fairly thin creamy-colored mixture was achieved. Speed-gel was then added, and the composition was treated to remove bubbles. The pH was adjusted to 6.8 with 30% NaOH. At about pH 6.0, the solution thickened became clear, and from this point to pH 6.8, it was a thick, amber-colored, homogenous gel.
Those skilled in the art will recognize that Ketoprofen topical patches are being extensively used for treatment of musculoskeletal pain. The patches have been shown to provide rapid and sustained delivery to underlying tissues without significantly increasing levels of drug concentration in the blood when compared to the traditional oral administration.

### Example 4

### Preparation of Another 5% Ketoprofen Composition

| | 30 gm |
|---|---|
| Ketoprofen | 1.5 gm |
| Lecithin organogel | 3.0 gm |
| Speed-gel | 30 gm |

Ketoprofen was added to 1\veen 80 and heated until a clear solution was achieved, then speed-gel was added.

### Example 5

### Preparation of Another 5% Ketoprofen Composition

| | 30 gm |
|---|---|
| Ketoprofen | 1.5 gm |
| Lecithin organogel | 7.0 gm |
| Speed-gel | 22 gm |

1. The ketoprofen was dissolved in lecithin organogel using moderate heat.
2. Speed-gel was added and the mixture was heated in a microwave to a clear gel.
3. The mixture was then set on a hot plate with stirring.
4. 30% NaOH was then added to bring the pH to 5.8-6.8 to form a gel.

### Example 6

### Preparation of 5% Ketoprofen, 2% Lidocaine Gel, Keto-Lido 5-2

| | 30 gm |
|---|---|
| Ketoprofen | 1.5 gm |
| Lidocaine base | 0.6 gm |
| Lecithin organogel | to wet |
| Speed-gel | 30 gm |

1. The ketoprofen and lidocaine were triturated to a fine powder.
2. The speed-gel was heated and added to the powders with stirring.
3. A few drops of lecithin organogel were added to the mixture to thicken.
4. The composition was dispensed into a jar for later use.

### Example 7

### Preparation of 5% Ketoprofen, 2% Lidocaine Gel, 0.5% Cyclobenzaprine Gel

| | 60 gm |
|---|---|
| Cyclobenzaprine | 0.3 gm |
| Polysorbate 80 | to wet |
| Keto-Lido 5-2 (Example 6) | 60 gm |

1. The cyclobenzaprine was moistened with polysorbate.
2. The mixture was brought to final weight by trituration with Keto-Lido 5-2 compound of Example 6.

### Example 8

### Preparation of 5% Ketoprofen, 2% Lidocaine Gel, 0.5% Cyclobenzaprine Gel

| | 60 gm |
|---|---|
| Ketoprofen | 1.3 gm |
| Lidicaine base | 1.3 gm |
| Cyclobenzaprine | 0.3 gm |
| L.O. | to wet |
| Speed -gel | 60 gm |

1. The ketoprofen, lidocaine and cyclobenzaprine were triturated to a fine powder.
2. The speed-gel was heated and added to the powders with stirring.
3. A few drops of lecithin organogel were added to the mixture to thicken.
4. The formulation was dispensed into a jar for later use.

### Example 9

### Preparation of Ketoprofen 10% Gel

| | 60 gm |
|---|---|
| Ketoprofen | 6 gm |
| L.O. | 7 gm |
| Speed -gel | 44 gm |
| L.I.D. oil or Polysorbate 80 | 2-3 ml to thicken |

1. L.O. was added to the ketoprofen and stirred to a smooth paste.
2. Speed-gel was added and stirred to smooth paste and heated until clear.
3. 2-3 ml of LID oil was then added.
4. The pH and thickness were then adjusted by titrating the pH to 5.9-6.8 with 30% NaOH.

### Example 10

### Preparation of Ketoprofen 10% Gel, Cyclobenzaprine 1% Gel

| | 30 gm | |
|---|---|---|
| Ketoprofen | 3 | gm |
| Cyclobenzaprine | 0.3 | gm |
| Speed -gel | 30 | gm |

1. The powders were triturated until fine.
2. The speed-gel was added and mixed until smooth.

### Example 11

### Preparation of 20% Ibuprofen Gel

A speed-gel containing ibuprofen was prepared without the need to add any additional surfactant because the ibuprofen itself acts as a surfactant. This formulation was prepared as follows:

| | 100 gm | |
|---|---|---|
| Ibuprofen | 20 | gm |
| L.O. | 25 | gm |
| Urea | 10 | gm |
| Water | 36 | gm |
| Benzyl Alcohol | 1 | ml |
| 30% NaOH | 5 | ml |

These reagents were mixed in the order listed above and brought to a pH of about 6.8. The color gel has a pleasant amber color and light and even consistency.

### Example 12

### Preparation of Trolamine Salicylate 10% Speed-Gel

The following analgesic topical solution was prepared:

| | 3.0 gm | |
|---|---|---|
| Salicylic Acid | 1.5 | gm |
| Trolamine | 1.5 | gm |
| Ethyl Alcohol (95%) | 1.0 | ml |
| Tween80 | 1 | gm |
| Speed-Gel | 16 | gm |
| L.O. | 9 | gm |

1. The salicylic acid, trolamine, alcohol and tween were combined, triturated and heated to form a clear solution.
2. The speed-gel was added.
3. The L.O. was added and the gel formed was dispensed into a container for later use.

### Example 13

### Preparation of Aminophylline 2% Speed-Gel

The following cream is prepared to reduce cellulite by transport of aminophylline (theophylline ethylenediamine, 2:1; theophylline is 3,7-dihydro-1,3-dimethyl-1H-purine-2,6-dione) across the skin and into a dipose tissue. Transdermal delivery of this active agent across the skin is known to achieve cellulite reduction as described in Clinical Thera-peutics 9 (No. 6):663-671, 1987.

| | | |
|---|---|---|
| Aminophylline | 2 | gm |
| L.O. | 10 | gm |
| Speed-Gel | 100 | gm |

The aminophylline is dissolved in heated L.O. and then mixed with the speed-gel to form a consistent and easily applied gel.

### Example 14

### Preparation of Capsaicin Speed-Gel

A speed-gel for relief of, for example, postherpetic neuralgia, is prepared as a 0.025 to 0.075% gel of capsaicin oleoresin:

| | | |
|---|---|---|
| Capsaicin | .025 | gm |
| L.O. | 1 | gm |
| Speed-Gel | 100 | gm |

The above examples of topical ointments are not inclusive of all possible drugs 110 that may be utilized in the transdermal delivery system kit 100, but rather an example of possible active agents 106 and inactive agents 110 that may be compounded.

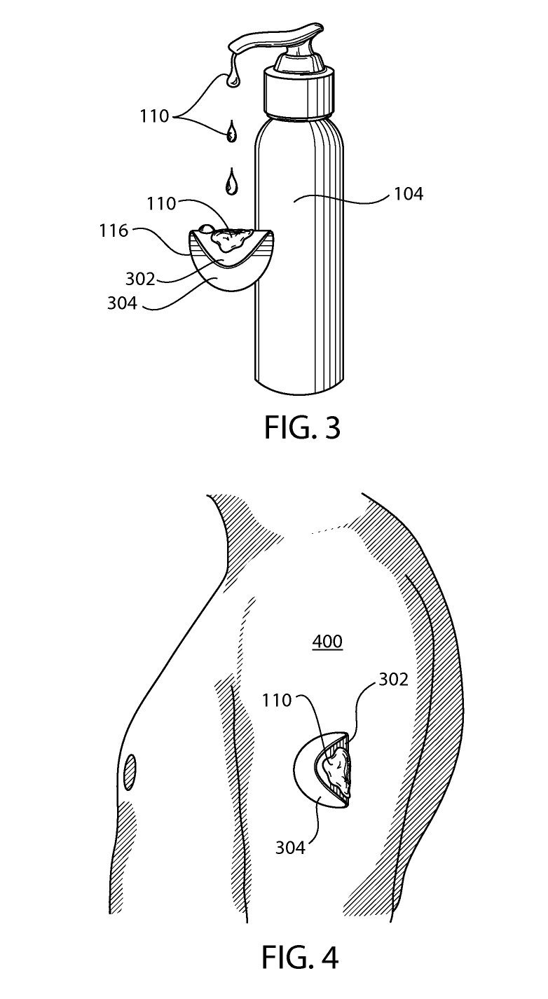

FIG. 3 presents a view of the topical contact surface 302 of an exemplary occlusive member 116 receiving an exemplary drug 110 from the drug dispensing device 104. In some embodiments, the transdermal delivery system kit 100 may further include at least one occlusive member 116 for actuating absorption of the drug 110 through the topical contact surface area 400 (shown in FIG. 4) and delivering a specific dose of the drug 110 through the topical contact surface area 400. Each occlusive member 116 may be efficacious for actuating absorption of the drug through the topical contact surface area 400. The absorption initiates through the stratum corneum, then through the epidermis, and finally to the dermal layer, before delivering a specific dose of the drug into the bloodstream, joint, or affected area. The occlusive member 116 may actuate absorption in a variety of ways. First, the occlusive member 116 may act as a reservoir for the drug, releasing the drug in a staged, time-release manner. In another embodiment, the occlusive member 116 acts as a cover over the skin 400. The covering effect may help to drive medication into the skin 400 with the help of body heat. It is well known in the art that retaining moisture from the top layer of the skin enhances absorption. In yet another system the occlusive member 116 simply acts as a cover to prevent the drug 110 from rubbing off.

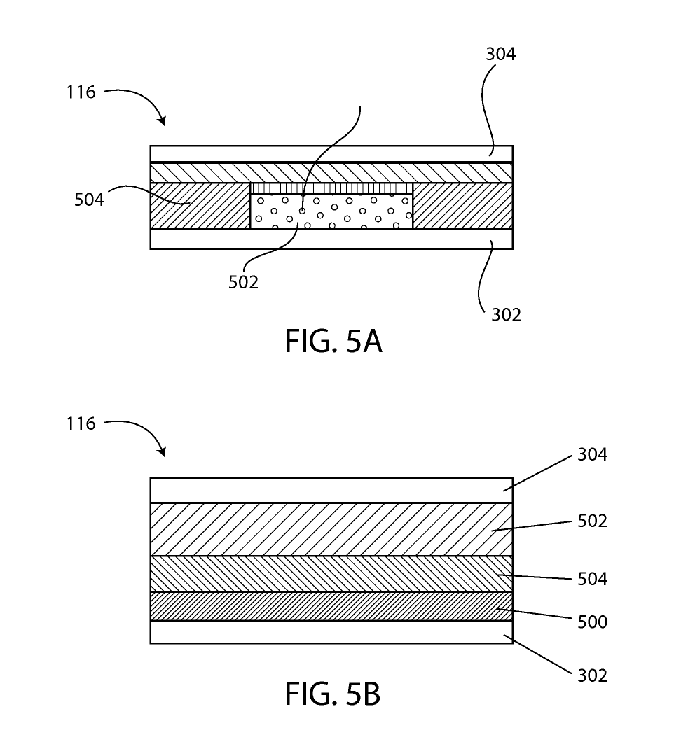

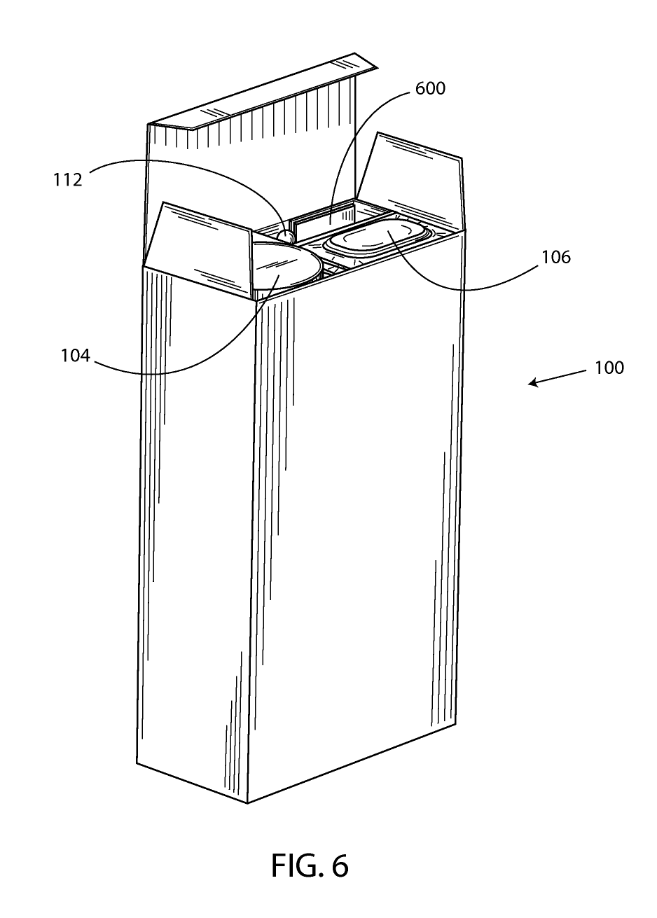

FIGS. 5A and 5B present sectioned views of an exemplary occlusive member 116, in accordance with an embodiment of the present invention. Each occlusive member 116 may include, without limitation, a transdermal delivery patch and a bandage. Each occlusive member 116 may include an impermeable external surface 304. The external surface 304 may be utilized to provide a surface for positioning and pressing the occlusive member 116 against the topical contact surface area 400. The at least one occlusive member 116 may include a topical contact surface 302. The topical contact surface 302 may be configured to maintain the drug 110 in contact with the topical contact surface area 400 (See FIG. 4). The topical contact surface 302 may include porosity sufficient for allowing the molecules of the drug 300 to pass through. The drug 300 may be dispensed onto the topical contact surface 302, and the topical contact surface 302 may be positioned over the desired topical contact surface area 400.

In some embodiments of the present invention, the topical contact surface 302 may include an adhesive 500 to stabilize and localize the occlusive member 116 on a specific topical contact surface area 400. In yet another embodiment, the occlusive member may include a drug reservoir 502 for containing a predetermined quantity of the drug 110 prior to the transdermal delivery. The drug reservoir 502 may be positioned between the topical contact surface 302 and the external surface 304. A rate control membrane 504 may position between the topical contact surface 302 and the drug reservoir 502 for regulating the diffusion of the drug 300 through the occlusive member 116. The rate control membrane 504 may include varying diffusion rates based on the dilution of the drug 110, the porosity of the rate control membrane 504, and the osmotic pressure. In additional embodiments, the adhesive 500 may also be utilized to securely join any of the components in the occlusive member. For example, without limitation, the adhesive 500 may stabilize and localize the occlusive member 116 on a specific topical contact surface area 400, or securely join the rate control member 504 to the topical contact surface 302. The topical contact surface 302 may then be maintained over the topical contact surface area 400 from a predetermined quantity of time, or until absorption of the drug 300 through the topical contact surface area 400 is complete.

Those skilled in the art, in light of the present teachings, will recognize that topical drugs may include messy, greasy characteristics that hinder transdermal delivery because of the difficulty in maintaining the drug 110 over a specific, desired topical contact surface area 400. The occlusive member 116 acts as a carrier to drive the drug 110 through the layers of the topical contact surface area 400, yet also acts to localize delivery of the drug 110 on a specific topical contact surface area 400. Another difficulty with transdermal delivery of the drug 110 is that the molecules of the drug 110 may be too large to pass through the topical contact surface area 400. Those skilled in the art recognize that only a limited number of drugs 110 are amenable to administration by a transdermal patch. With current delivery methods, successful transdermal drugs 110 may include molecular masses that are only up to a few hundred Daltons, such as octanol-water partition coefficients that heavily favor lipids and require doses of milligrams per day or less. However, with the occlusive member 116 of the present invention, the external surface 304 may be pressed against the topical contact surface area 400 to force the molecules of the drug through to the dermis, where systemic absorption can occur.

In one alternative embodiment of the present invention, the occlusive member 116 may include two separate components; a drug saturated member for absorbing the drug 110 prior to use, and an adhesive member for adhering the drug saturated member onto the topical contact surface area 400 when needed. The drug saturated member provides the transdermal delivery functions. The drug saturated member may be efficacious for absorbing the drug 110 after compounding. Those skilled in the art will recognize that the drug saturated member may include sufficient porosity to absorb the drug 110, and then deliver the drug 110 onto the topical contact surface area 400. After the drug saturated member has absorbed a sufficient quantity of the drug 110, the drug saturated member may then be temporarily stored in the transdermal delivery system kit 100 until needed. Unlike the prior embodiment, the patient is not required to dispense the drug 110 onto the occlusive member 116, since the drug 110 has been predispensed onto the drug saturated member. In some embodiments, the drug saturated member requires an additional component to adhere to the topical contact surface area 400. An adhesive member acts to overlay the drug saturated member, holding the drug saturated member in position over the topical contact surface area 400.

In yet another alternative embodiment, the occlusive member 116 may include the drug 110 applied onto the topical contact surface 302 without requiring compounding. In this embodiment, the topical contact surface 302 of the occlusive member 116 is presaturated with the drug 110. In this type of occlusive member 116, the topical contact surface 302 not only serves to adhere the various layers together, along with the entire system to the topical contact surface area 400, but is also responsible to release the drug 110 through the topical contact surface area 400. The topical contact surface 302 may be surrounded by a temporary liner and a backing. In yet another alternative embodiment, the occlusive member 116 may include a separate drug layer. The drug layer is a liquid compartment containing a drug solution or suspension separated by the topical contact surface 302. In this type of transdermal delivery, the rate of release for the drug 110 is zero order. In yet another alternative embodiment, the topical contact surface 302 not only serves to adhere the various layers together, but also to release a vapor embodiment of the drug 110. Vapor patches have been known to release essential oils for up to 6 hours and may be used in cases of decongestion mainly.

In yet another alternative embodiment, the drug 110 may be applied directly to the topical contact surface area 400 without the utilization of the occlusive member 116. The drug 110 may include a topical gel ointment that may be dispensed directly onto the affected area.

A first aspect of the present invention provides a transdermal delivery system kit 100 including at least one drug dispensing device 104, the at least one drug dispensing device 104 including at least one inactive agent 101, 102, at least one container 108, e.g., individual packets, or other similar holders. The number of containers 108 can be any number, depending on the number of active and inactive ingredients provided in the particular kit 100. In one embodiment, the dispensing device 104 is provided with a base/inactive agent and only a single container 108 is provided with the kit, the single container 108 containing at least on active agent that is added to the dispensing device 104 for mixing. A mixing member 112, instructions 114 for compounding the at least one active agent 106, 107 with the at least one inactive agent 101, 102 to form the drug 110, and at least one occlusive member 116 for actuating absorption of the drug 110 through a topical contact surface area 400. The at least one occlusive member 116 includes a topical contact surface 302, the topical contact surface 302 is configured to maintain the drug 300 in contact with the topical contact surface area 400. The at least one occlusive member 116 further includes an external surface 304.

In a further aspect, the transdermal delivery system kit 100 is configured to store myriad classes of drugs 110, including, without limitation, transdermal delivery system drugs.

In another aspect, instructions 114 are included in the transdermal delivery system kit 100 to direct the appropriate compounding procedure for the drug 300.

In another aspect, the occlusive member 116 is actuated to administer the drug 110 with thermal energy, such as body heat.

In another aspect, the occlusive member 116 utilizes time-release to gradually administer the drug 110 through the topical contact surface area 400.

In another aspect, the occlusive member 116 includes an adhesive to localize delivery of the drug 110 on a specific topical contact surface area 400.

One benefit of the transdermal delivery system kit 100 is that it allows a pharmacist to compound a specific topical drug 110 for a patient with predetermined quantities of the at least one active agent and the at least one inactive agent so that the pharmacist is not required to measure the agents.

Another benefit of the transdermal delivery system kit 100 is that a patient can dispense the correct dosage of the drug 110 onto the topical contact surface 302 of the occlusive member 116 because of the calibrated pump that dispenses a predetermined quantity of the drug 110 on the occlusive member 116.

Another benefit of the transdermal delivery system kit 100 is that the occlusive member 116 enhances the absorption of particular types of drugs 110.

Another benefit of the transdermal delivery system kit 100 is that the occlusive member 116 allows for bypassing the absorption of the drug 110 through the stomach. This drug delivery route will decrease stomach irritation, esophageal and stomach lining deterioration issues, and ulcers.

Another benefit is that since the active drug 110 may possess a low oral bioavailability, it will pass the first pass metabolism through the liver if administered transdermally; thereby enhancing the bioavailability of the drug 110.

Another benefit is that the occlusive member 116 acts as a barrier between the drug and the exterior of the patient; thereby inhibiting contact between people in proximity to the patient and the drug 110.

Another benefit is the ability to meet the patient's specific medicinal needs by compounding a customized drug formula tailored for the patient.

Another benefit is that time and expense are saved for both the pharmacist and the patient.

The transdermal delivery system kit 100 is designed to provide a prepackaged environment that is efficacious for the accurate compounding of at least one active agent and at least one inactive agent into at least one drug dispensing device 104. The resultant drug 110 can be applied directly to the treatment region 400 or dispensed onto at least one occlusive member 116 for systemic transdermal administration. Details of the compounding and administration of the drug 110 onto an occlusive member 116 are illustrated in FIGS. 2 through 4. In operation, the initial drug 110 compounding may be performed by a pharmacist who utilizes the transdermal delivery system kit 100 to compound the drug 110 from each active agent 106, 107 and each inactive agent 101, 102 in the transdermal delivery system kit 100. A variety of tools may be utilized to facilitate the compounding process, including, without limitation, a mixing member 112, instructions 114 for compounding, pH actuators, and solvents. Initially, the at least one inactive agent 110, base, solvent, pH actuator, and the like are premeasured and prepackaged into at least one drug dispensing device 104. Each active agent 106, 107 may also be prepackaged and premeasured into at least one container 108, such as a glass vial. The agents 101, 102, 106, 107 are then mixed with the mixing member 112 (or shaken) in the drug dispensing device 104 to form the desired drug 110. At this point, additional components of the transdermal delivery system kit 100 may be utilized to administer the drug 110. The patient may then dispense the drug 110 from the at least one drug dispensing device 104. The drug 110 may be dispensed onto each occlusive member 116 from the transdermal delivery system kit 100. Each occlusive member 116 may be efficacious for actuating absorption of the drug 110 through the stratum corneum, then through the epidermis, and finally to the dermal layer before delivering a specific dose of the drug into the bloodstream, joint, or affected area. The drug 110 may be dispensed onto the topical contact surface 302, and the dermal contact surface 302 may be positioned over the desired topical contact surface area 400. The topical contact surface 302 may then be maintained over the topical contact surface area 400 for a predetermined quantity of time, or until absorption of the drug 110 through the topical contact surface area 400 is complete. After administration, the transdermal delivery system kit 100 may be reused. The pharmacist may compound a different drug 110 from the same kit, only varying the process, or negating some of the agents 101, 102, 106, 107 to formulate the desired drug 110.

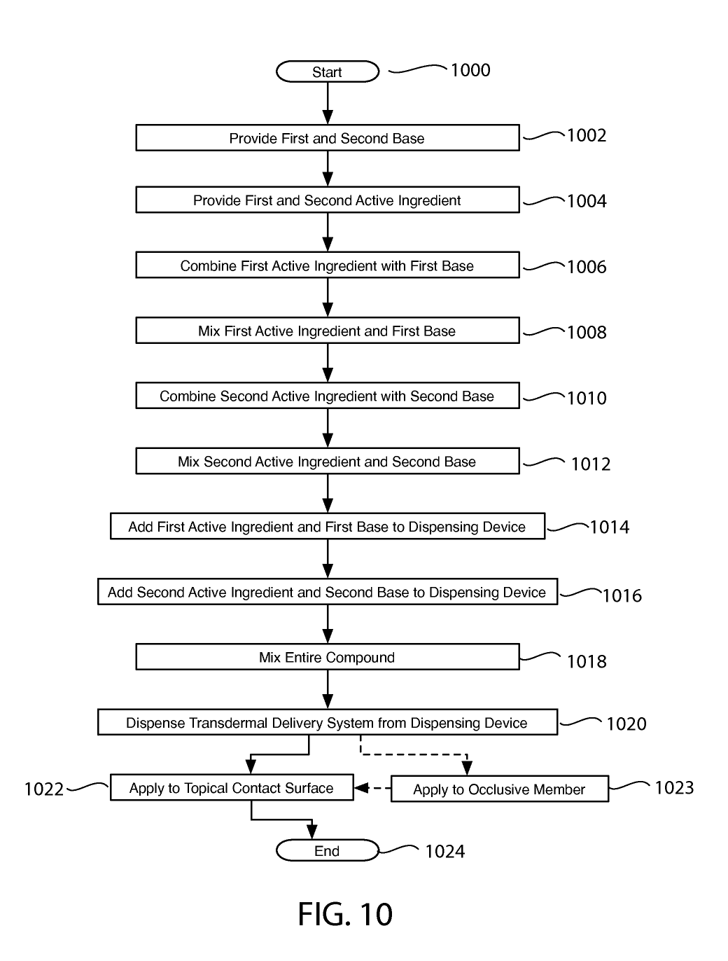

FIG. 10 provides a process flow diagram illustrating a method for utilizing one embodiment of the present invention. Process illustrated in FIG. 10 is based upon a kit 100 having two different bases, one for combination with water soluble active agents and one for combination with lipid soluble active agents. When combined, they will form a novel transdermal-deliver system in accordance with the present invention.

The process beings at step 1000 and moves directly to step 1002 where a first base 101 and a second base 102 are provided as part of a transdermal delivery system kit 100. An exemplary first base 101 is an “LID” (Lecithin, Isopropyl, Docusate) lipid or fat soluble (lipophilic) vehicle. An exemplary second base 102 is a “PWU” (Purified Water, Urea) water soluble (hydrophilic) vehicle. The PWU base does not necessarily have to contain urea. In step 1004, a first active ingredient 106 and a second active ingredient 107 are added to the transdermal delivery system kit 100. An exemplary first active ingredient 106 is an active pharmaceutical ingredient that is lipid soluble. An exemplary second active ingredient 107 is an active pharmaceutical ingredient that is water soluble.

In step 1006, the first active ingredient 106 is added to the container 108 holding the first base 101. The two are mixed together in step 1008. In a further step, 1010, the second active ingredient 107 is added to the container 108 holding the second base 102. The two are mixed together in step 1012.

Next, in step 1014, the combined first base 101 and first active ingredient 106 are added to the drug dispensing device 104. In step 1016, the combined second base 102 and second active ingredient 107 are also added to the drug dispensing device 104. In step 1018, the compound is mixed together, e.g., by stirring or shaking. The compound is now ready for dispensing.

In step 1020, the compounded drug 110 is dispensed from the drug dispensing device 104 and, in step 1022, applied to the topical contact surface area 400 of a user. In an optional step, 1024, the compounded drug 110 is covered by an occlusive member 116. Optionally, in step 1023, the compounded drug 110 is dispensed from the drug dispensing device 104 and applied to an occlusive member 116, which is then applied to the topical contact surface area 400 of a user. The process ends at step 1024.

Of course, the novel transdermal delivery system kit 100 does not require both an LID and PWU base and may only include one of the two, depending on the particular active ingredient provided with the transdermal delivery system kit 100. Similarly, the transdermal delivery system kit 100 does not require both a lipid soluble active ingredient and a water soluble active ingredient. In addition, the transdermal delivery system kit 100 may include multiple LID bases and/or multiple PWU bases as well as multiple lipid soluble active ingredients and multiple water soluble active ingredients. The ingredients can be selected in accordance with the particular drug desired.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A transdermal delivery system kit for compounding a drug and administering the drug through transdermal delivery, the kit comprising:
at least one drug dispensing device;
a lipophilic base;
a hydrophilic base;
a lipid-soluble active ingredient;
a water-soluble active ingredient;
a first container for mixing the lipophilic base with the lipid-soluble active ingredient to
form a first compound;
a second container for mixing the hydrophilic base with the water-soluble active ingredient to form a second compound;
a mixing member; and instructions for:
compounding the lipophilic base with the lipid-soluble active ingredient to form the first compound;
compounding the hydrophilic base with the water-soluble active ingredient to form the second compound; and
mixing the first compound and the second compound to form a transdermal pharmaceutical delivery system capable of delivering a drug to the dermal layer of the skin.

2. The transdermal delivery system kit according to claim 1, further comprising:
at least one occlusive member for actuating absorption of the drug through a topical contact surface area of the recipient of the drug.

3. The transdermal delivery system kit according to claim 2, wherein the at least one occlusive member comprises a transdermal delivery-patch.

4. The transdermal delivery system kit according to claim 2, wherein the at least one occlusive member includes a topical contact surface, the topical contact surface of the occlusive member being configured to maintain the drug in contact with the topical contact surface area.

5. The transdermal delivery system kit according to claim 1, wherein the at least one drug dispensing device comprises a pump dispenser.

6. The transdermal delivery system kit according to claim 1, wherein the at least one drug dispensing device is calibrated to dispense a predetermined quantity of the drug.

7. The transdermal delivery system kit according to claim 1, wherein at least one of the lipid-soluble active ingredient, the water-soluble active ingredient, the lipophilic base, and the hydrophilic base are provided in a quantity that can be used to form the drug without the need for measurement by a compounding user.

8. The transdermal delivery system kit according to claim 1, wherein at least one of the lipid-soluble active ingredient and the water-soluble active ingredient comprise an analgesic, an anti-inflammatory, a muscle relaxer, a biologically active protein, a cellulite reducer, a substance P antagonist, or an antineoplastic compound.

9. The transdermal delivery system kit according to claim 1, wherein at least one of the lipophilic base and the hydrophilic base comprise a biocompatible organic solvent, a polar lipid, a surfactant, water, urea, a suspending agent, or an anti-foaming agent.

10. The transdermal delivery kit according to claim 1, wherein at least one of the lipophilic base and the hydrophilic base comprise a permeation or penetration enhancer for transdermal drug delivery.

11-18. (canceled)

19. A transdermal delivery system kit for compounding a drug and administering the drug through a transdermal delivery system, the kit comprising:
at least one container and at least one mixing member for mixing at least one inactive agent with at least one active agent in at least one drug dispensing device to form the drug;
an occlusive member having sufficient porosity to absorb the drug and become a drug saturated member which is temporarily stored in the transdermal delivery system kit until needed;
an adhesive member for positioning and maintaining the drug saturated member on a topical contact surface; and
instructions for directing said compounding of said drug;
thereby actuating absorption of the drug through the topical contact surface area.

20. The transdermal delivery system kit according to claim 19, wherein the occlusive member and topical contact surface provide a transdermal delivery system for actuating absorption of the drug through the topical contact surface area.

21. A transdermal delivery system kit for compounding a drug and administering the drug through a transdermal delivery system, the kit comprising:
at least one first container and at least one first mixing member for mixing at least one first inactive agent with at least one first active agent in at least one drug dispensing device to form a first compound;
at least one second container and at least one second mixing member for mixing at least one second inactive agent with at least one second active agent in at least one drug dispensing device to form a second compound;
an occlusive member having sufficient porosity to absorb the first compound, the second compound and admixtures thereof to become a drug saturated member which is temporarily stored in the transdermal delivery system kit until needed;
an adhesive member for positioning and maintaining the drug saturated member on a topical contact surface area;
thereby actuating absorption of the drug through the topical contact surface area, said drug saturated member and adhesive member forming a transdermal delivery system;
and
instructions for directing said compounding of each said first and second compounds.

22. The kit of claim 21, wherein:
said at least one first inactive agent is a lipophilic base;
said at least one first active agent is lipid-soluble;
said at least one second inactive agent is a hydrophilic base; and
said at least one second active agent is water-soluble.

23. The kit of claim 21, wherein each said drug dispensing device is a calibrated pump constructed and arranged for dispensing a predetermined quantity of each said first and second compounds.

24. The kit of claim 21, wherein said at least one first active agent and said at least one second active agent is selected from the group consisting of an analgesic, an anti-inflammatory, a muscle relaxer, a biologically active protein, a cellulite reducer, a substance P antagonist, an antineoplastic compound and combinations thereof.

25. The kit of claim 21, wherein said at least one first inactive agent and said at least one second inactive agent is selected from the group consisting of a biocompatible organic solvent, a polar lipid, a surfactant, water, urea, a suspending agent, an anti-foaming agent, and combinations thereof.
